(19) United States
(12) Patent Application Publication
(10) Pub. No.: US 2025/0283227 A1
(43) Pub. Date: Sep. 11, 2025

(54) Apparatus, system and method for producing hydrogen peroxide, hydrocarbon(s) and syngas

(71) Applicant: The University of Sydney, Sydney (AU)

(72) Inventors: Patrick Cullen, Sydney (AU); Renwu Zhou, Sydney (AU); Tianqi Zhang, Sydney (AU); Josip Knezevic, Sydney (AU)

(21) Appl. No.: 18/683,051

(22) PCT Filed: Aug. 9, 2022
(86) PCT No.: PCT/AU2022/050868

§ 371 Date: Feb. 12, 2024

(30) Foreign Application Priority Data
Aug. 13, 2021 (AU) 2021902528

(51) Int. Cl. C25B 1/50; C25B 1/23; C25B 1/30; C25B 3/07; C25B 9/17; C25B 11/037; C25B 11/077; C25B 15/08

(52) U.S. Cl. CPC C25B 1/50; C25B 1/23; C25B 1/30; C25B 3/07; C25B 9/17; C25B 11/037; C25B 11/077; C25B 15/08

(57) ABSTRACT

A plasma-bubble reactor, reactor system and method for producing hydrogen peroxide (H₂O₂), one or more hydrocarbon(s), and syngas is disclosed. The reactor comprises a vessel configured to hold a liquid; and a plasma generating means, in association with the vessel, configured to receive an input feed comprising carbon dioxide (CO₂) gas and generate a plasma from the CO₂ gas to produce an activated CO₂ gas encapsulated within a plurality of bubbles formed in the liquid, wherein the activated CO₂ gas reacts with water (H₂O) at a plasma-liquid interface formed between the bubbles and the surrounding liquid to produce hydrogen peroxide (H₂O₂), one or more hydrocarbon(s) and syngas.

### FIELD OF THE INVENTION

The present invention relates to an apparatus, system and method for producing hydrogen peroxide, one or more hydrocarbon(s) and syngas.

The invention has been developed primarily for use in the production of useful precursors for the chemical industry and will be described hereinafter with reference to this application. However, it will be appreciated that the invention is not limited to this particular field of use.

### BACKGROUND OF THE INVENTION

The global mean concentration of carbon dioxide (CO₂) in the biosphere has increased from 280 ppm in the mid-18th century and hit 416 ppm in 2021, mainly due to anthropogenic activities, especially the burning of fossil fuels such as coal, petroleum and natural gas. The increasing concentration of CO₂ has caused a series of problems, including global warming, desertification, and ocean acidification. In response to these issues, the development of innovative technologies to significantly reduce CO₂ emissions has received noticeable attention, and substantial progress has been made to this end in recent years, especially under the impetus of nations to achieve global emission reduction targets and the Paris Agreement commitments.

The major challenge of converting CO₂ lies in overcoming the high stability of the CO₂ molecule—in other words, breaking the linear and centrosymmetric double bonds (O═C=O). Conventional strategies have been investigated and developed for CO₂ conversion to value-added chemicals or renewable fuels (e.g., CO, CH₄ and liquid chemicals), mainly including thermal catalysis, electrocatalysis and photocatalysis processes. However, these methods-based productions of fuels and chemicals are energy-intensive, and more importantly some processes consume large amounts of valuable high-purity hydrogen.

In recent years, non-thermal plasma (NTP), generated by applying electrical energy to a feeding gas, has been shown as an effective tool to provide an attractive solution for activating inert CO₂ molecules into more reactive, vibrationally or electronically excited states to promote dissociation of CO₂ molecules, and thus, facilitate the breaking of the highly stable double bonds (O═C=O).

The use of NTP has led to the efficient conversion of CO₂ into higher value chemicals and fuels. The energetic electrons produced in NTP have an average electron temperature of 1-10 eV and are capable of activating CO₂ molecules by ionisation, excitation and dissociation, creating an avalanche of reactive species (e.g., excited atoms, ions, molecules and radicals) that can initiate and propagate chemical reactions. The major challenges of converting CO₂ using NTP are in improving the energy efficiency, increasing the competitiveness of the plasma process and selectively generating the chemical compounds.

The present invention seeks to provide an apparatus, system and method for producing hydrogen peroxide, one or more hydrocarbon(s) and syngas, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the present invention there is provided a plasma-bubble reactor, comprising:
a vessel configured to hold a liquid; and
a plasma generating means, in association with the vessel, configured to receive an input feed comprising carbon dioxide (CO₂) gas and generate a plasma from the CO₂ gas to produce an activated CO₂ gas encapsulated within a plurality of bubbles formed in the liquid, wherein the activated CO₂ gas reacts with water (H₂O) at a plasma-liquid interface formed between the bubbles and the surrounding liquid to produce hydrogen peroxide (H₂O₂), one or more hydrocarbon(s) and syngas.

In one embodiment, the plasma generating means comprises two electrodes, wherein at least one of the two electrodes is a high voltage (HV) electrode at least partially immersed within the liquid, and configured to generate an electric discharge through the liquid for activating the CO₂ gas encapsulated within the bubbles when a potential difference is applied across the electrodes.

In one embodiment, each of the two electrodes is at least partially immersed within the liquid.

In one embodiment, each of the two electrodes is an HV electrode at least partially immersed within the liquid.

In one embodiment, the other of the two electrodes is a ground electrode electrically connected to an external wall of the vessel.

Preferably, the HV electrode is partially enclosed within a tube defining a gas passage extending partially along a length of the HV electrode, wherein the tube is in fluid communication with the input feed and configured with one or more outlets at a lower portion thereof to allow the activated CO₂ gas encapsulated within the bubbles to exit therefrom into the liquid in the vessel.

Suitably, the two electrodes are electrically connected to a DC or AC power supply.

In one embodiment, the reactor further comprises a means for adjusting the vertical position of the HV electrode relative to the tube to generate longer plasma streamers within the gas passage.

Preferably, the vertical position of the HV electrode is adjustable relative to the tube within a range of about 0 mm to about 60 mm.

In one embodiment, the tube of the HV electrode comprises a catalytically active material for catalysing the reaction between the activated CO gas and H₂O.

In one form, the catalytically active material comprises a plurality of aluminium oxide beads.

In some embodiments, the one or more hydrocarbon(s) are selected from the group consisting of formic acid, acetic acid and oxalic acid.

In a preferred embodiment, the hydrocarbon(s) is oxalic acid.

According to a second aspect of the present invention there is provided a reactor system comprising:
two or more plasma-bubble reactors, wherein each plasma-bubble reactor comprises:
   a vessel configured to hold a liquid, wherein each vessel comprises a plurality of ports;
   a plasma generating means, in association with the vessel, configured to receive an input feed comprising carbon dioxide (CO₂) gas and generate a plasma from the CO₂ gas to produce an activated CO₂ gas encapsulated within a plurality of bubbles formed in the liquid, wherein the activated CO₂ gas reacts with water (H₂O) at a plasma-liquid interface formed between the bubbles and the surrounding liquid to produce hydrogen peroxide (H₂O₂), one or more hydrocarbon(s) and syngas; and
   a plurality of fluid conduits, wherein each fluid conduit is configured to operably couple adjacent plasma-bubble reactors together via corresponding ports to enable fluid communication of one or more of CO₂ gas, H₂O₂, one or more hydrocarbon(s), syngas and/or H₂O therebetween.

In one embodiment, the plasma generating means comprises two electrodes, wherein at least one of the two electrodes is a high voltage (HV) electrode at least partially immersed within the liquid, and configured to generate an electric discharge through the liquid for activating the CO₂ gas encapsulated within the bubbles when a potential difference is applied across the electrodes.

In one embodiment, the other of the two electrodes is a ground electrode electrically connected to an external wall of the vessel.

Preferably, the reactor system further comprises a pump for fluidly communicating water from a water supply to the vessel of one of the two or more plasma-bubble reactors.

Preferably, the reactor system further comprises a compressor for enhancing the flow of CO₂ gas from the input feed to the vessel of one of the two or more plasma-bubble reactors.

Preferably, the reactor system further comprises a flowmeter disposed in line between the compressor and the vessel of the one plasma-bubble reactor to monitor the flow rate of the CO₂ gas.

Preferably, the reactor system further comprises a liquid receiver for receiving H₂O₂ from the vessel of one of the two or more plasma-bubble reactors.

In some embodiments, the one or more hydrocarbon(s) are selected from the group consisting of formic acid, acetic acid and oxalic acid.

In a preferred embodiment, the hydrocarbon(s) is oxalic acid.

According to a third aspect of the present invention there is provided a method for producing hydrogen peroxide (H₂O₂), one or more hydrocarbon(s) and syngas, the method comprising the steps of:
generating plasma from an input feed comprising carbon dioxide (CO₂) gas to produce an activated CO₂ gas encapsulated within a plurality of bubbles formed in liquid; and
reacting the activated CO gas with water (H₂O) at a plasma-liquid interface formed between the bubbles and the surrounding liquid to produce hydrogen peroxide (H₂O₂), one or more hydrocarbon(s) and syngas.

In one embodiment, the plasma is generated by applying a potential difference across two electrodes, wherein at least one of the two electrodes is a high voltage (HV) electrode at least partially immersed within the liquid, and configured to generate an electric discharge through the liquid for activating the CO: gas encapsulated within the bubbles.

In one embodiment, the electric discharge is a pulsed discharge.

Preferably, the potential difference falls within a range of between about 5 kV and about 100 kV.

Preferably, the liquid is an aqueous medium.

Preferably, the aqueous medium comprises an electrolyte.

Preferably, the reaction is carried out in a vessel substantially under atmospheric pressure and room temperature.

In one embodiment, the input feed comprises a mixture of the CO₂ gas and a second gas.

Preferably, the second gas is selected from the group consisting of carbon monoxide (CO), water vapour/steam (H₂O), methane (CH₄), hydrogen (H₂), nitrogen (Na) and any mixture thereof.

In one embodiment, the HV electrode is partially enclosed within a tube defining a gas passage extending partially along a length of the HV electrode, the method further comprising the step of:
adjusting the vertical position of the HV electrode relative to the vertical position of the tube to generate longer plasma streamers within the gas passage.

Preferably, the vertical position of the HV electrode is adjustable relative to the vertical position of the tube within a range of about 0 mm to about 60 mm.

In one embodiment, the method further comprises the step of:
catalysing the reaction between the activated CO₂ gas and H₂O.

In some embodiments, the one or more hydrocarbon(s) are selected from the group consisting of formic acid, acetic acid and oxalic acid.

In a preferred embodiment, the hydrocarbon(s) is oxalic acid.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a schematic representation of a plasma-bubble reactor according to a preferred embodiment of the present invention, configured to activate carbon dioxide (CO₂) using plasma for subsequent reaction with water (H₂O) to produce hydrogen peroxide (H₂O₂), oxalic acid (C₂H₂O₄), and syngas (CO, H₂ and O₂);

FIG. 2 shows schematic representations of four (4) different configurations of the plasma-bubble reactor of FIG. 1, including (a) reactor, (b) a reactor with an adjustable High Voltage (HV) electrode height (h), (c) a reactor equipped with an HV electrode modified with a catalyst, and (d) a reactor equipped with two (2) HV electrodes;

FIG. 3 shows a plot showing the gas ratio in the gas phase output from reactions involving the plasma-bubble reactors of FIGS. 2(a), 2(b) and 2(c) when the plasma-driven process employs an input feed of CO₂ gas;

FIG. 4 shows: (A) a renewable energy-driven plasma microbubble reactor for generating underwater microbubbles for the electrified reduction of CO₂ into green fuels, (B) a plot of H₂O₂ concentration (mg L⁻¹) in solution versus time (min) after plasma discharge, (C) shows a plot of oxalic acid production rate (mg h⁻¹) versus reduced flow rate (sccm) of CO₂, and (D) shows a plot of H₂O₂ production rate (mg h⁻¹) versus reduced flow rate (sccm) of CO₂;

FIG. 5 shows the electrical characteristics (5.8 kV, 1500 Hz, 35.5 W, 10 sccm CO₂) of the plasma microbubble reactor of FIG. 4A;

FIG. 6 shows the effect of pH on CO₂ plasma discharge at different initial pH values on the production rate (mg h⁻¹) of: (A) formic acid and acetic acid concentration (as quantified by NMR, where (B) shows an NMR spectrum of the control group), (C) oxalic acid concentration (as quantified by oxalate assay), and (D) H₂O₂ concentration (as measured by the titanium (IV) sulfate method);

FIG. 7 shows the UV-Vis standard curve of H₂O₂ concentration (mg L⁻¹) by titanium (IV) sulfate method at 410 nm;

FIG. 8 shows a plot of oxalic acid concentration (ppm) versus time (min) showing the dependence of oxalic acid production using the plasma microbubble reactor of FIG. 4A, operating with the same electrical characteristics (5.8 KV, 1500 Hz, 35.5 W, 10 sccm CO₂) outline in FIG. 5;

FIG. 9 shows: (A) a plot showing the gas ratio (%) in the gas phase output from reactions involving the plasma-bubble reactor of FIG. 4A when the plasma-driven process employs an input feed of CO₂ gas, and (B) a plot showing a decrease in the CO₂ conversion rate (%) (diamonds) with increasing CO₂ flow rate (scom) and increasing CO energy efficiency (g kWh⁻¹) (circles); and

FIG. 10 shows a schematic representation of a plasma-bubble reactor system according to another preferred embodiment of the present invention, comprising three plasma-bubble reactors, operably coupled together via a plurality of fluid conduits to enable fluid communication of one or more of CO₂ gas, H₂O₂, one or more hydrocarbon(s), syngas and/or H₂O therebetween.

### DETAILED DESCRIPTION OF SPECIFIC EMBODIMENTS

It should be noted in the following description that like or the same reference numerals in different embodiments denote the same or similar features.

The present invention is predicated on the finding of a process for converting carbon dioxide (CO₂) gas into the important and industrially useful products of hydrogen peroxide (H₂O₂) and syngas-products more commonly associated with the dry reforming and anthraquinone processes, respectively, and one or more hydrocarbon(s) including but not limited to formic acid (CH₂O₂), acetic acid (CH₃COOH) and oxalic acid (C₂H₂O₄).

Here, the inventors have found that by employing the use of non-thermal plasma (NTP), it is possible to overcome the activation energy gap associated with the highly stable O═C=O bond of CO₂ to generate active species that can be used to react with water (H₂O) molecules. Here, the H₂O is used as a green reducing agent and oxygen receiver, producing H₂O₂ as a product.

The two key steps in this process mainly include plasma pre-activation and interactions between H₂O and the plasma-activated CO₂ gas. Various species (including electrons, ions, radicals, molecular fragments) with different energy levels are present in the plasma ionised gas.

Different from thermal plasma (equilibrium plasma) with high bulk gas temperature (typically higher than 5×10³ K), NTP operates in a more ambient temperature condition, but it gives enough energies to activate stable molecules and drive the reaction across the energy gap, with excellent selectivity of products and high energy efficiency.

This process is thus in stark contrast to the conventional industrial process, whereby the reduction of CO₂ typically uses valuable hydrogen or methane gases, which inevitably consumes more energy.

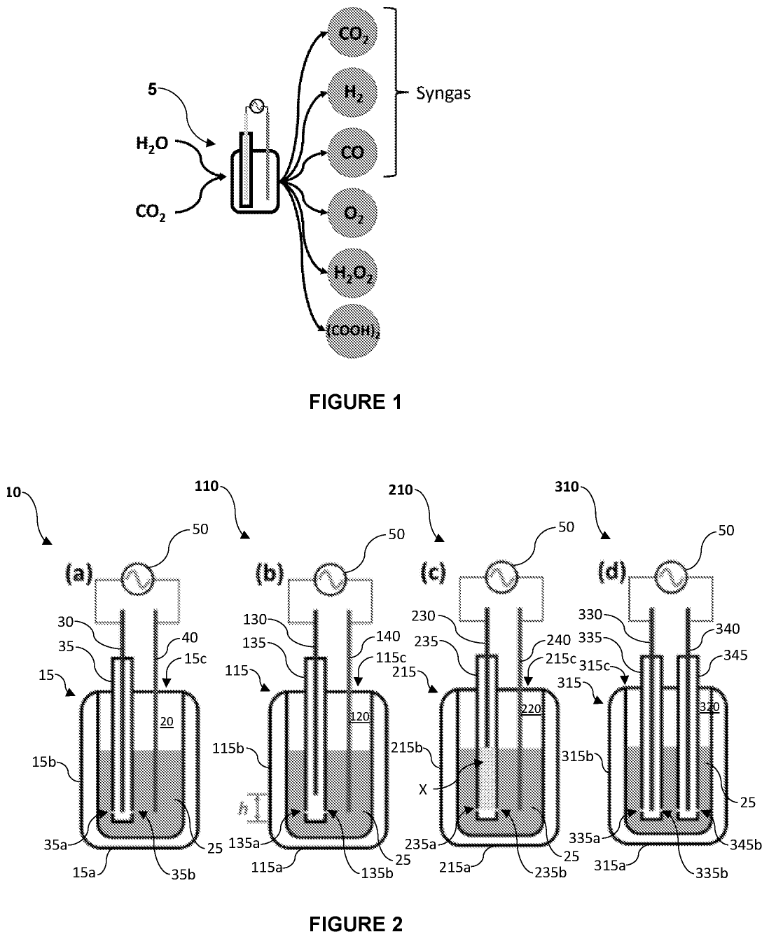

FIG. 1 shows a schematic representation of a simplified plasma-bubble reactor 5 according to a preferred embodiment of the present invention, that is configured to activate carbon dioxide (CO₂) using non-thermal plasma (NTP), generated by a High Voltage (HV) electrode immersed in a liquid medium comprising water (H₂O), wherein the plasma-activated CO₂ reacts with H₂O to produce H₂O₂, oxalic acid (C₂H₂O₄) and syngas (CO, H₂, O₂).

Without being bound by any one particular theory, the inventors believe that ground state carbon dioxide molecules (CO₂) can be activated under the strongly alternating electric field associated with the plasma, to form excited state molecules (CO₂*, CO*) and release atomic oxygen atoms (O). These reactive species can further react with the water molecules in the liquid medium to form H₂O₂, CO, O₂, Ha and one or more hydrocarbon(s) including but not limited to formic acid (CH₂O₂), acetic acid (CH₃COOH) and oxalic acid (C₂H₂O₄).

By controlling the reactor design, and the plasma conditions, and coupling with a catalyst, it is possible to have the chemical outputs as three valuable chemicals/fuels; H₂O₂ and said hydrocarbon(s) in the liquid phase and syngas (H₂+CO+O₂) in the gas phase.

What follows is a detailed description of four (4) different configurations of the plasma-bubble reactor 5 in FIG. 1, a reactor system 400 configured for continuous production of H₂O₂, said hydrocarbon(s) and syngas, and a method for the production thereof.

### Plasma-Bubble Reactors

FIG. 2 shows schematic representations of four (4) different configurations of the plasma-bubble reactor 5 of FIG. 1, including (a) single reactor 10, (b) a single reactor 110 with an adjustable High Voltage (HV) electrode height (h), (c) a single reactor 210 equipped with an HV electrode modified with a catalyst, and (d) a double reactor 310 equipped with an HV electrode and a Low Voltage (LV) electrode.

The following description outlines the structural details of each of the four (4) different configurations.

In the simplest configuration, as represented in FIG. 2(a), the plasma-bubble reactor 10 comprises a vessel 15 that comprises a base 15a and a wall 15b upstanding from the base 15b to define a cavity 20 configured to hold a liquid medium 25 and an opening 15c at an upper portion of the vessel 15.

The plasma-bubble reactor 10 further comprises a plasma generating means in the form of two electrodes 30, 40 that are located within the cavity 20 of the vessel 15, via the opening 15c, and partially immersed in the liquid medium 25.

The two electrodes 30, 40 are electrically connected to an AC power supply 50. Although it will be appreciated by persons of skill in the relevant art that in an alternative embodiment, the two electrodes 30, 40 may be electrically connected to a DC power supply (not shown).

The first electrode 30 is a High Voltage (HV) electrode (or cathode), while the second electrode 40 is a counter electrode (or anode).

The HV electrode 30 is partially enclosed within a quartz tube 35 defining a gas passage extending partially along a length of the HV electrode 30. The tube 35 comprises a gas inlet (not shown) at an upper portion thereof that is configured to receive an input feed comprising carbon dioxide (CO₂) gas from a CO₂ gas supply (not shown), and one or more gas outlets 35a, 35b at a lower portion thereof, wherein the lower portion of the HV electrode 30 is fully immersed within the liquid medium 25.

### Alternative Arrangements

The components of the three other plasma-bubble reactor configurations 110, 210, 310 of FIG. 2(b), (c), (d) are labelled in a similar manner to those in FIG. 2(a), where a prefix of “1”, “2” or “3” is employed before the reference numeral of each component to signify that the component relates to the corresponding plasma-bubble reactor 110, 210, 310 of FIG. 2(b), (c), (d), respectively.

In FIG. 2(b), the plasma-bubble reactor 110 further comprises a means (not shown) for adjusting the vertical position “h” of the HV electrode 130 relative to the tube 135 within which it is partially enclosed. In one embodiment, the height of the HV electrode 130 relative to the tube 135 can be adjusted within a range of about 0 mm to about 60 mm. By virtue of this arrangement, it is possible to increase or decrease the length of the plasma streamers within the gas passage of the tube 135, thereby providing a means by which to increase or decrease the degree of gas ionisation/excitation.

In FIG. 2(c), the tube 235 of the HV electrode 230 further comprises a catalytically active material for catalysing the reaction between the activated CO₂ gas and H₂O.

In one embodiment, the catalytically active material takes the form of a plurality of particles, beads, pellets or flakes that are supported within the tube 235. In this arrangement, the particles, beads, pellets or flakes act as a supported catalyst and are typically manufactured from a polymer, ceramic, glass or metal oxide.

In one particularly preferred embodiment, the catalytically active material comprises a plurality of Al₂O₃ beads.

In FIG. 2(d), the two electrodes partially immersed within the liquid medium 25 in the vessel 315 consists of an HV electrode 330 powering a first reactor and a low voltage (LV) electrode 340 powering a second reactor. The inventors consider that the use of a second reactor driven by the low voltage electrode 340 will amount to an increased rate of production of H₂O₂ and syngas and improve energy efficiency.

### Method

A method for producing hydrogen peroxide (H₂O₂), one or more hydrocarbon(s) and syngas using the single reactor of FIG. 2(a) as a general guide will now be described.

According to a first step of the method, a potential difference is applied across the two electrodes 30, 40 causing the HV electrode 30 to generate an electric discharge within the tube 35. The electric discharge generates a plasma from the CO₂ gas that has been fed into the tube 35 via the input feed to produce an activated CO₂ gas. The activated CO gas exits the tube 35 via the gas outlets 35a, 35b and forms a plurality of bubbles in the liquid medium 25, which for the purpose of this embodiment is an aqueous liquid medium comprising an electrolyte.

In one embodiment, the electric discharge is a pulsed discharge, that is repeatedly applied at a frequency that falls with a range of about 50 Hz and about 10 MHz. Under such conditions, the potential difference that is to be applied across the two electrodes 30, 40 typically falls within a range of between about 1 KV and about 100 kV.

According to a second step of the method, the activated CO₂ gas encapsulated within the bubbles produces a plurality of excited molecules selected from the group consisting of CO₂*, CO* and oxygen atoms (O). These excited molecules then react with the water (H₂O) in the aqueous liquid medium 25 at a plasma-liquid interface formed between the bubbles and the surrounding liquid medium 25 to produce a liquid phase comprising at least hydrogen peroxide (H₂O₂) and one or more hydrocarbons including but not limited to formic acid (CH₂O₂), acetic acid (CH₃COOH) and oxalic acid (C₂H₂O₄), and a gas phase comprising syngas.

In one embodiment, the reaction is carried out substantially under atmospheric pressure and at room temperature, although it will be appreciated by persons of skill in the relevant art that altering one or both of these parameters can be used as a means by which to increase or decrease the rate of conversion of CO₂ gas to H₂O₂, said one or more hydrocarbon(s) and syngas.

### Results

The inventors have found that by altering one or more of the plasma input voltage (amplitude and pulse width), the frequency (electric discharge and resonance), the gas flow rate of the input feed of CO₂ gas, and/or the liquid flow rate of H₂O, it becomes possible to alter the rate of conversion of CO₂ gas to H₂O₂, said one or more hydrocarbon(s) and syngas.

For instance, the inventors have found that the input voltage changes the ratio of plasma species, gives differentiated reaction selectivity, and power efficiency. While the frequency changes the total power input and plasma density without changing the power efficiency.

Without wishing to be bound by any one particular theory, the inventors believe that the reaction at the plasma-liquid interface can be contributed to two main pathways. One pathway involves water sputtering and splitting by electron bombardment, followed by combination of two OH radicals produced by the electron bombardment, which contributes to a relatively small fraction of H₂O₂ formation. The majority of H₂O₂ comes from the interaction between the H₂O molecules and the atomic oxygen produced from the CO₂ gas. The second pathway contributes to the production of the small amount of Hz in the liquid aqueous medium due to the combination of two H radicals produced by electron bombardment.

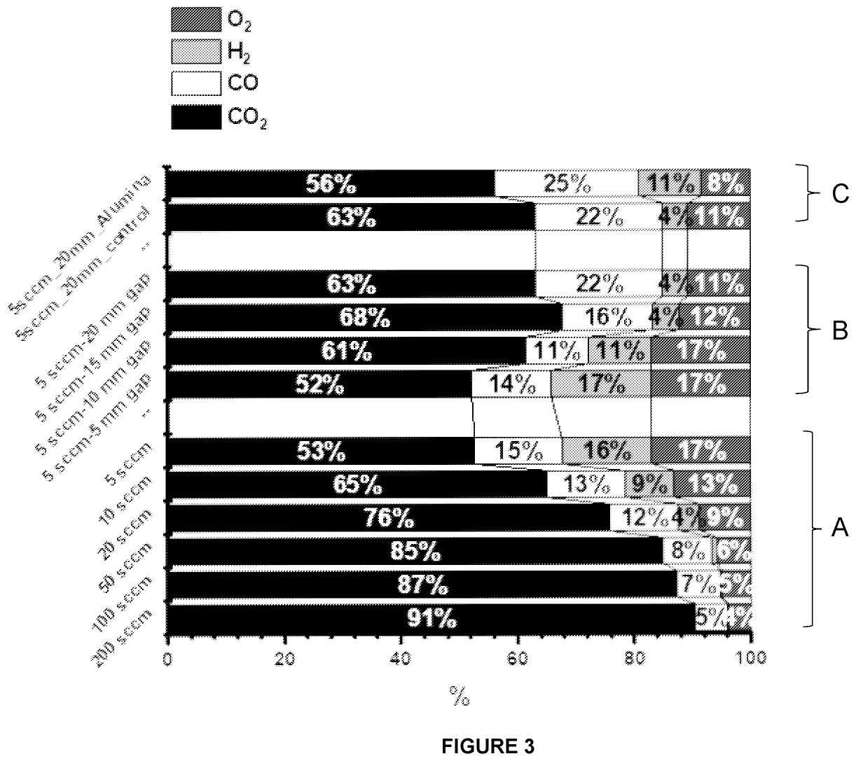

FIG. 3 shows a plot showing the gas ratio in the gas phase output stream from the plasma-bubble reactors 10, 110, 210 of FIGS. 2(a), 2(b) and 2(c) when the plasma-driven process employs an input feed of CO₂ gas.

### CO₂ Gas Flow Rate

For instance, when the flow rate of CO₂ gas from the input feed to the vessel 15 of the plasma-bubble reactor 10 shown in FIG. 2(a) is decreased from 200 sccm to 5 sccm, the corresponding results in FIG. 3(A) show that a greater proportion of the input feed CO₂ gas undergoes plasma-activation (and subsequent reaction with H₂O), as evidenced by a decrease in the overall volume of CO₂ gas present in the output stream from 91% to 53%.

The results in FIG. 3(A) also reveal a three-fold increase in the volume of CO gas in the output stream, as well as significant increases in the volumes of both Ha and O₂.

Based on the observed results, it is clear that by reducing the flow rate of the input CO₂ gas, it becomes possible to increase the volumes of the individual syngas components that may be produced.

Gap Size (“h”)

In the case of the plasma-bubble reactor 110 shown in FIG. 2(b), when the HV electrode 130 is raised within the tube 135 from 5 mm to 20 mm at a fixed flow rate of CO₂ gas (5 sccm), the corresponding results in FIG. 3(B) show that the proportion of input feed CO₂ gas that undergoes plasma-activation (and subsequent reaction with H₂O) remains largely the same, irrespective of the size of the gap (“h”). Although, there is an approximate 1.5-fold increase in the volume of CO that is produced as the gap size (“h”) is increased from 5 mm to 20 mm. By contrast, the volume of Hz gas in the output stream shows a significant decrease from 17% to 4% for the same change in gap size (“h”), while the volume of O₂ shows only a slight decrease from 17% to 11%.

A corresponding study to determine how the gap size (“h”) impacts on the production of hydrogen peroxide (H₂O₂) reveals that when the flow of CO₂ gas is maintained at the same 5 sccm flow rate, and then the size of the gap (“h”) is increased from 0 mm to 30 mm, the volume of H₂O₂ produced decreases (see Table 1).

TABLE 1

| | Gap Size (“h”) | | | |
|---|---|---|---|---|
| | 0 mm | | | |
| | (Control) | 10 mm | 20 mm | 30 mm |
| Input Power (W) | 110 | 110 | 110 | 110 |
| Discharge Power (W) | 31.9 | 29.8 | 27.0 | 28.0 |
| CO₂ flow rate (sccm) | 5 | 5 | 5 | 5 |
| Time of operation (min) | 20 | 20 | 20 | 20 |
| Volume of solution (mL) | 150 | 150 | 150 | 150 |
| H₂O₂ concentration (mM) | 8.7 | 4.7 | 2.0 | 1.5 |
| H₂O₂ Production rate (mmol/h) | 3.9 | 2.1 | 0.9 | 0.7 |
| H₂O₂ Energy efficiency (g/kWh) | 4.1 | 2.0 | 1.0 | 0.7 |

Based on the observed results, it is clear that by altering the gap size “h”), it becomes possible to selectively control the volumes of H₂O₂, oxalic acid (C₂H₂O₄), and the individual syngas components that may be produced.

### Catalysis

In the case of the plasma-bubble reactor 210 shown in FIG. 2(c), when the tube 235 of the HV electrode 230 is modified with a plurality of Al₂O₃ beads (labelled “x”) supported within the tube 235 by a mesh plate and glass wool, the corresponding results in FIG. 3(C) show that when the CO₂ gas flow rate and gap size (“h”) are maintained at 5 sccm and 20 mm, respectively, there is a noticeable decrease in the volume CO₂ gas present in the output stream of around 7% (when compared to a control experiment involving plasma activation only with no beads), coupled with a 3% increase in the volume of CO and a 7% increase in the volume of H₂.

### Example

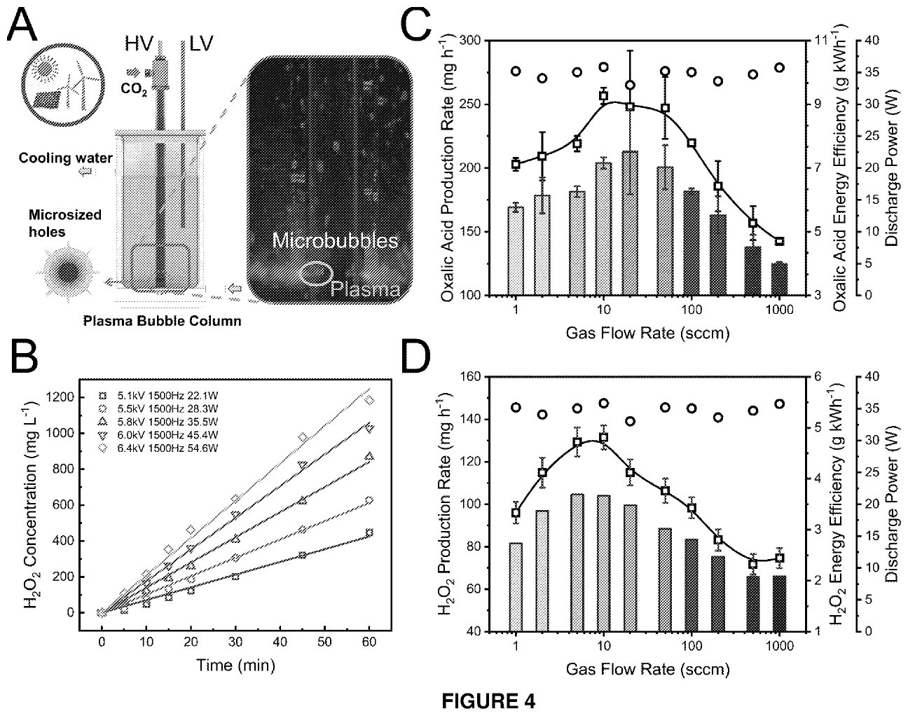

As illustrated in FIG. 4A, a renewable energy-driven plasma microbubble reactor is developed to generate underwater microbubbles for the electrified reduction of carbon dioxide into green fuels. The microsized holes distributed on the column not only serve as the channels for the microplasma generation, but also produce small microbubbles transferring reactive plasma species.

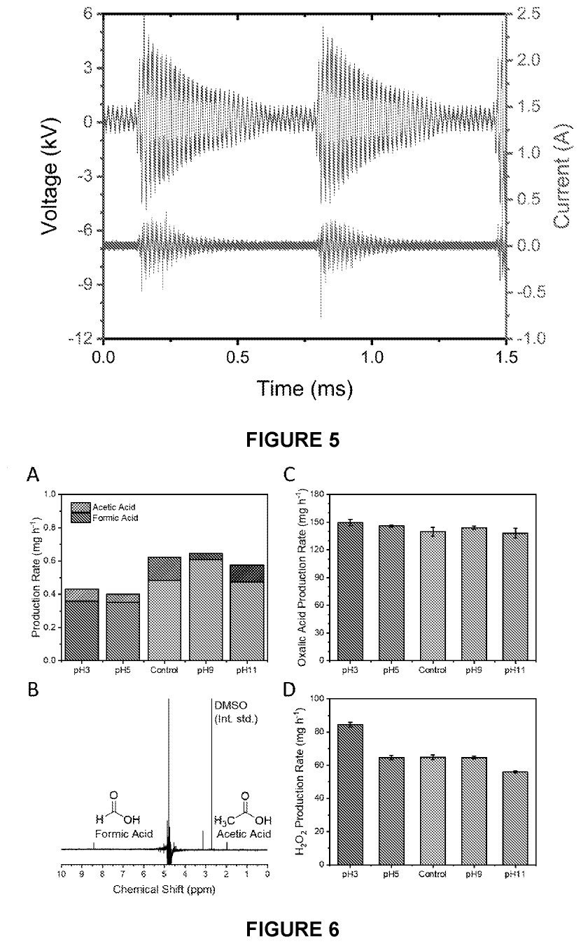

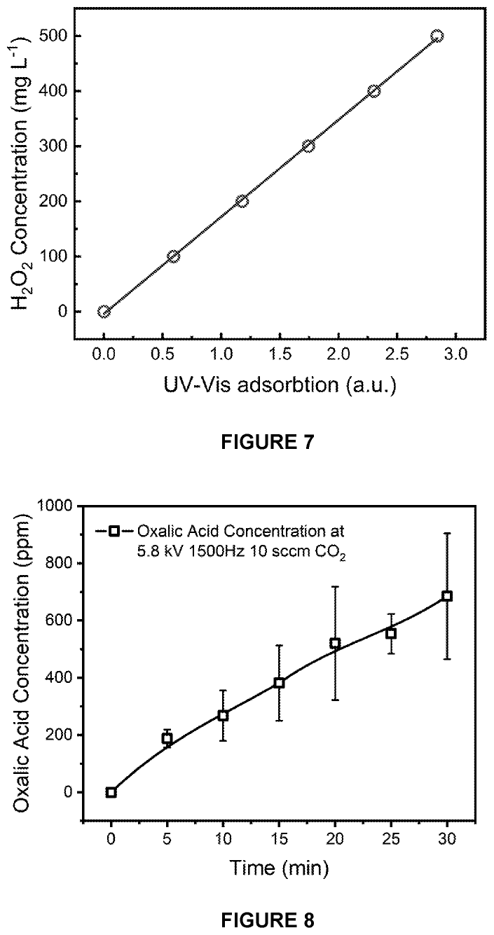

The electrical characteristics of the plasma microbubble reactor (5.8 kV, 1500 Hz, 35.5 W. 10 sccm CO₂) are shown in FIG. 5.

CO₂ is used as the feed gas with different flow rates ranging from 1 to 1000 sccm.

Once CO₂ is fed through the plasma column and the discharge is generated, plasma generated species will be delivered by bubbles, then transported into and/or react with water molecules in the aqueous media. These bubbles are expected to serve as unique micro-reactors with a large gas-liquid interface, which facilitates the CO₂ conversion at the plasma-liquid interface.

Aqueous H₂O₂ generated from the CO₂ plasma-water system was quantitatively analysed using titanium (IV) sulfate with the addition of NaN₃.

FIG. 4B shows the time-dependent concentration of H₂O₂ in solution with respect to plasma discharge. As shown in this figure, the H₂O₂ concentration was linearly enhanced with a higher voltage amplitude for driving the plasma bubble column, with a 190.8 mg L⁻¹ concentration of H₂O₂ obtained after 30 min plasma treatment at 200 V.

Further optimization of the CO₂ plasma-water system involved varying the flow rate of CO₂ and solution pH (FIG. 4C and FIG. 4D).

For instance, as shown in FIG. 4D, the H₂O₂ production rate (mg h⁻¹) initially Increased with a reduced flow rate (sccm) of CO₂, and then decreased when further reducing the gas flow. It should be noted that the discharge power of the CO₂ plasma discharge remains almost unchanged no matter how much the gas flow changes.

As shown in FIG. 4D, the production rate (mg h-1) for oxalic acid was essentially double that observed for H₂O₂ (see FIG. 4C).

The highest production rate of H₂O₂ was achieved at the flow rate of 10 sccm, with a relatively high energy efficiency of ˜3.6 g kWh⁻¹.

Liquid hydrocarbon fuels produced in the CO₂ plasma-water system are quantified using a cryogenic NMR spectroscopy and a UV-vis spectroscopy coupled with a colorimetric assay.

NMR spectra of the treated solution after the CO₂ plasma discharge clearly demonstrate the presence of formic acid (CH₂O₂) and acetic acid (CH₃COOH), as shown in FIG. 6A.

Another C₂-hydrocarbon species (oxalic acid) is quantified using an enzymatic chemical assay, and similar trends of its production rate (mg h 1) and energy efficiency with H₂O₂ as a function of CO₂ flow rate (sccm) are shown in FIG. 4D. It should be noted that compared with the oxalic acid content with the order of several hundreds of ppms, the yields of formic acid and acetic acid produced in the CO₂ plasma-water system are quite low, with only several ppms.

Without being bound by any one particular theory, the inventors believe that one possible reaction pathway for the production of such liquid hydrocarbon fuels takes place via the combination of vibrationally excited CO₂* species generated in the dielectric barrier discharge (DBD) section, which subsequently react with H₂O-dissociated H radicals at the plasma-liquid interface.

As described above, the syngas produced as a gaseous stream in the CO₂ plasma-water system, mainly includes carbon monoxide (CO), carbon dioxide (CO₂) and hydrogen (H₂), which can be widely used as an intermediate resource for the production of, for example, hydrogen (H₂), ammonia (NH₃), methanol (CH₃OH), and other synthetic hydrocarbon fuels.

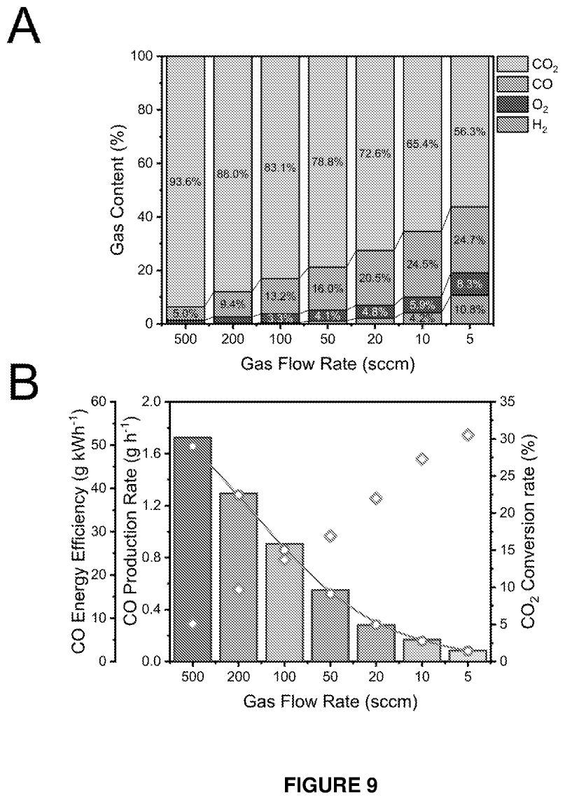

FIG. 9 shows how the ratio of the products in the output gas phase can be tuned by the gas flow rate. Clearly, the conversion of CO₂ gas showed a downward trend with a rise of total gas flow rate (sccm), and the highest conversion of CO₂ was obtained at the gas flow rate of 5 sccm. This may be attributed to the fact that the long residence time of the gaseous molecules in the discharge area contributes to the strong collisions between energetic electrons and CO₂ molecules, favouring the CO₂ conversion. It also coincides with the results in the CO₂ dissociation and CO₂ hydrogenation in other DBD discharges.

### Materials and Methods

### Plasma Parameters and Characterization

A digital oscilloscope (RIGOL DS6104) was employed to record the applied voltage and current with a high voltage probe (Tektronics P6015A) and a current probe (Pearson 4100), respectively. The discharge power was calculated based on previously reported studies.

H₂O₂ Quantification

The H₂O₂ concentration was measured using the titanium sulfate method. When titanium (IV) ions Ti⁴⁺ reacted with hydrogen peroxide, a yellow-coloured complex is formed with a UV-Vis absorbance at 410 nm (Ti⁴⁺+H₂O₂+2H₂O→H₂TiO₄+4H⁺). Shimadzu (Japan) UV-2600i UV-Vis spectrophotometer was used for colorimetry analysis.

### Gas Chromatography (GC) Quantification

Gaseous product compounds were quantified by Shimadzu (Australia) Nexis GC-2030 equipped with FID, TCD and ECD detectors using C18 and 5 Å columns. Hydrogen (Ha) and oxygen (O₂) were quantified by TCD detector. Carbon monoxide (CO) and carbon dioxide (CO₂) were quantified by FID detector with a methanizer. Standard gas (Supelco® 501743) was purchased from Sigma-Aldrich (Australia), with gas composition of CO₂ (7%), CO (15%), O₂ (4%), and CH₄ (4.5%).

### Oxalic Acid Quantification

An oxalate assay kit (MAK315) from Sigma-Aldrich (Australia) was used to quantify oxalic acid generated by CO₂ plasma discharge. The use of the assay kit followed the product information.

The calculations were based on the following equation:

$$[ OXALATE ] = \frac{{OD}_{SAMPLE} - {OD}_{BLANK}}{{OD}_{STANDARD} - {OD}_{BLANK}} \times 500 \times D ( µmol \text{/L} )$$

where D represents the dilution rate and OD represents the optical density value collected by UV-Vis spectroscopy at the wavelength of 595 nm.

### NMR Quantification

Formic acid and acetic acid were quantified by nuclear magnetic resonance (NMR) spectroscopy using a Bruker (Germany) AVIII 600 MHz NMR Spectrometer equipped with a cryogenic triple nucleus probe head.

All NMR samples were prepared by mixing with D₂O and dimethyl sulfoxide (DMSO, internal standard) solution to a final 10% D₂O and 10 ppm DMSO concentration.

The 1D-1 H-NMR spectra at 600 MHz were recorded using the zgesgppe pulse sequence to suppress the water signal. The acquisitions were maintained at 298 K using a spectral width of 14 ppm, a time domain data size of 67k, 8 dummy scans, and 32 scans.

### Chemicals

Sulfuric acid (H₂SO₄, ACS reagent, 95.0%-98.0%), titanium (IV) chloride (TiCl₄, ReagentPlus®, 99.9% trace metals basis), hydrogen peroxide solution (30% w/w in H₂O, contains stabilizer), isotope carbon dioxide (C¹⁸O₂, 95% atom ¹⁸O), deuterium oxide (D₂O, 99.9 atom % D), dimethyl sulfoxide (DMSO, ACS reagent, ≥99.9%), sodium azide (NaN₃, ≥99.0%), oxalate assay kit (MAK315) were purchased from Sigma-Aldrich (Australia) without further purification. Carbon dioxide (CO₂, high purity grade, 99.99%) gas cylinder was purchased from BOC Gas (Australia).

### Reactor System

The inventors have found that by operably coupling together two or more plasma-bubble reactors, it becomes possible to realise a reactor system that enables the continuous production of hydrogen peroxide (H₂O₂), one or more hydrocarbon(s) including but not limited to formic acid (CH₂O₂), acetic acid (CH₃COOH) and oxalic acid (C₂H₂O₄), and syngas when a continuous feed of carbon dioxide (CO₂) and water (H₂O) is supplied to the system.

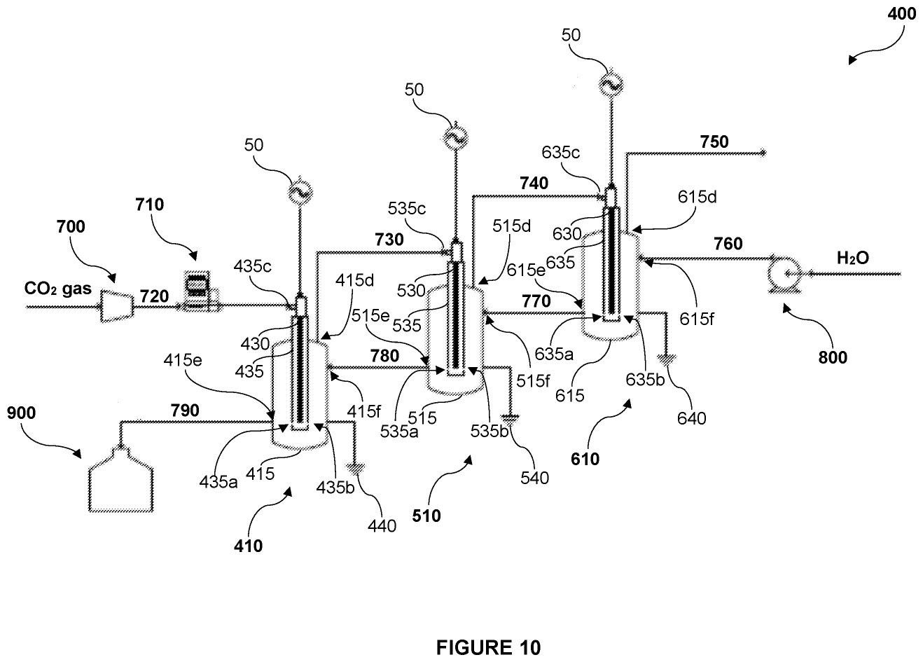

For instance, FIG. 10 shows a schematic representation of a reactor system 400 that comprises three plasma-bubble reactors 410, 510, 610, operably coupled together via a series of fluid conduits to enable fluid communication of one or more of CO₂ gas, H₂O₂, said one or more hydrocarbon(s), syngas and/or H₂O therebetween.

As shown in FIG. 10, the three plasma-bubble reactors 410, 510, 610 are slightly different to those shown in FIG. 2. For instance, and with reference to the first plasma-bubble reactor 410, the plasma generating means comprises a single HV electrode 430 partially immersed within the liquid in the vessel 415, and a ground electrode 440 that is electrically connected to an external wall of the vessel 415.

To facilitate the operable coupling between the plasma-bubble reactors 410, 510, 610, each of the corresponding vessels 415, 515, 615 comprises a plurality of ports that can be connected to a corresponding fluid conduit to enable the fluid communication of CO₂ gas, H₂O₂, syngas and/or H₂O from one vessel to the next.

In addition to the three plasma-bubble reactors 410, 510, 610, the reactor system 400 comprises a pump 800 for fluidly communicating water (H₂O) from a water supply (not shown) to the vessel 615 of the nearest (third) plasma-bubble reactor 610, a compressor 700 and a flowmeter 710 disposed in line between the input feed (not shown) of CO₂ gas and the vessel 415 of the nearest (first) plasma-bubble reactor 410 to, respectively, enhance the flow of CO₂ gas from the input feed to the vessel 415 and monitor the flow rate, and lastly, a liquid receiver 900 for receiving the H₂O₂ produced by all three plasma-bubble reactors 410, 510, 610.

In use, H₂O from the water supply is fluidly communicated, aided by the pressure applied by the pump 800, along conduit 760 to port 615f of the vessel 615 of the third plasma-bubble reactor 610. As the vessel 615 fills, the H₂O level rises until it reaches the level of port 615e. At this point, the pressure applied by the pump 800 drives the flow of H₂O along conduit 770 to port 515f of the vessel 515 of the central (second) plasma-bubble reactor 510. Similarly, once the H₂O level reaches the level of port 515e in this vessel 515, H₂O is then fluidly communicated along conduit 780 to port 415f of the vessel 415 of the first plasma-bubble reactor 410.

Once each of the three vessels 415, 515, 615 comprises a sufficient volume of H₂O, a stream of CO₂ gas is then fluidly communicated along conduit 720 from the input feed directly to the tube 435 of the HV electrode 430 partially immersed in the H₂O in the vessel 415 of the first plasma-bubble reactor 410.

A potential difference is then applied across the two electrodes 430, 440 of the first plasma-bubble reactor 410 to generate a plasma from the CO₂ gas within the tube 435. The activated CO₂ gas produced as a result then exits the tube 435 via the outlets 435a, 435b into the liquid medium in the vessel 415 in the form of a plurality of bubbles encapsulating the activated CO₂ gas.

The excited molecules (CO₂*, CO*, O) associated with the activated CO₂ gas encapsulated within the bubbles then react with the water (H₂O) in the vessel 415 at a plasma-liquid interface formed between the bubbles and the surrounding H₂O to produce a liquid phase comprising at least hydrogen peroxide (H₂O₂) and one or more of said hydrocarbon(s), and a gas phase comprising syngas.

The H₂O₂ that is produced is driven by the pressure applied by the pump 800 to exit port 415e of the vessel 415, where it is then fluidly communicated along conduit 790 to the liquid receiver 900. At the same time, the syngas, and any non-activated CO₂ gas remaining from the reaction, is driven by the flow of CO₂ gas from the input feed, aided by the compressor 700, to exit port 415d of the vessel 415 and fluidly communicated along conduit 730 to port 535c of the tube 535 of the HV electrode 530 partially immersed in the H₂O in the vessel 515 of the second plasma-bubble reactor 510.

A potential difference is then applied across the two electrodes 530, 540 of the second plasma-bubble reactor 510 to generate a plasma from the non-activated CO₂ gas within the tube 535. The activated CO₂ gas produced as a result then exits the outlets 535a, 535b of the tube 535 encapsulated within a plurality of bubbles to react with the H₂O in the vessel 515 at the plasma-liquid interface formed between the bubbles and the H₂O to produce more H₂O₂ and more syngas.

The H₂O₂, in combination with any H₂O in the vessel 515, is then driven by the pressure applied by the pump 800 to exit port 515e and fluidly communicated along conduit 780 to the vessel 415 of the first plasma-bubble reactor 410, where it is subsequently combined with any H₂O₂ produced by the first plasma-bubble reactor 410, in combination with any H₂O in the vessel 415, and then fluidly communicated along conduit 790 to the liquid receiver 900. The syngas, and any non-activated CO₂ gas remaining from the reaction, is driven by the flow of CO₂ gas from the input feed to exit port 515d and fluidly communicated along conduit 740 to the port 635c of the tube 635 of the HV electrode 630 partially immersed in the H₂O in the vessel 615 of the third plasma-bubble reactor 610.

There, the non-activated CO₂ gas is activated by the plasma generated in the tube 635 when a potential difference is applied across the two electrodes 630, 640 The activated CO₂ gas produced as a result, together with any syngas from the previous reactions, then exits the outlets 635a, 635b of the tube 635 in the form of bubbles. The excited molecules (CO₂*, CO*, O) associated with the activated CO₂ gas encapsulated within the bubbles then react with the water (H₂O) in the vessel 615 at a plasma-liquid interface formed between the bubbles and the surrounding H₂O to produce more H₂O₂ and more syngas.

The syngas produced in the vessel 615, together with any syngas produced by the first and second plasma-bubble reactors 410, 510 that is also present in the vessel 615, is driven by the positive pressure applied by the compressor 700 to exit port 615d where it is then fluidly communicated along conduit 750 to a gas collecting vessel (not shown).

While the H₂O₂, in combination with any H₂O in the vessel 615, is driven by the pressure applied by the pump 800 to exit port 615e to be fluidly communicated along conduit 770 to port 515f of the vessel 515 of the second plasma-bubble reactor 510, which in turn, will be fluidly communicated, together with any H₂O₂ produced by the second plasma-bubble reactor 510, along conduit 780 to port 415f of the vessel 415 of the first plasma-bubble reactor 410, before finally being fluidly communicated, together with any H₂O₂ produced by the first plasma-bubble generator 410, along conduit 790 to the liquid receiver 900.

By virtue of the above arrangement, the inventors have identified a reactor system 400 that enables the continuous production of hydrogen peroxide (H₂O₂) and syngas when an input feed of carbon dioxide (CO₂) and water (H₂O) is continually supplied to the system 400.

### Advantages

The present invention provides a number of advantages, including, but not limited to:

The realisation of a scalable “green” technology to generate industrially important products (H₂O₂ and syngas).

The realisation of a means by which to utilise carbon dioxide (CO₂) gas, that in turn, could help to reduce the amount of (CO₂) gas present in the environment.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

### OTHER EMBODIMENTS

While the preferred embodiments of the invention described above relate to the use of pure carbon dioxide (CO₂) as the input feed gas, it will be appreciated by persons of ordinary skill in the relevant art that the plasma-driven process may also employ CO₂ gas in combination with a second gas.

For instance, the input feed may comprise the use of mixtures of CO₂/CO, CO₂/H₂O (g), CO₂/CH₄ and CO₂/H₂ as an inlet gas.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the chemical industry.

1. A plasma-bubble reactor, comprising:
a vessel configured to hold a liquid; and
a plasma generating means, in association with the vessel, configured to receive an input feed comprising carbon dioxide (CO₂) gas and generate a plasma from the CO₂ gas to produce an activated CO₂ gas encapsulated within a plurality of bubbles formed in the liquid, wherein the activated CO₂ gas reacts with water (H₂O) at a plasma-liquid interface formed between the bubbles and the surrounding liquid to produce hydrogen peroxide (H₂O₂), one or more hydrocarbon(s) and syngas.

2. A reactor according to claim 1, wherein the plasma generating means comprises two electrodes, wherein at least one of the two electrodes is a high voltage (HV) electrode at least partially immersed within the liquid, and configured to generate an electric discharge through the liquid for activating the CO₂ gas encapsulated within the bubbles when a potential difference is applied across the electrodes.

3. A reactor according to claim 2, wherein each of the two electrodes is at least partially immersed within the liquid.

4. A reactor according to claim 3, wherein each of the two electrodes is an HV electrode at least partially immersed within the liquid.

5. A reactor according to claim 2, wherein the other of the two electrodes is a ground electrode electrically connected to an external wall of the vessel.

6. A reactor according to claim 2, wherein the HV electrode is partially enclosed within a tube defining a gas passage extending partially along a length of the HV electrode, wherein the tube is in fluid communication with the input feed and configured with one or more outlets at a lower portion thereof to allow the activated CO₂ gas encapsulated within the bubbles to exit therefrom into the liquid in the vessel.

7. (canceled)

8. A reactor according to claim 4, further comprising a means for adjusting the vertical position of the HV electrode relative to the tube to generate longer plasma streamers within the gas passage, wherein the vertical position of the HV electrode is adjustable relative to the tube within a range of about 0 mm to about 60 mm.

9. (canceled)

10. A reactor according to claim 6, wherein the tube of the HV electrode comprises a catalytically active material for catalysing the reaction between the activated CO₂ gas and H₂O, wherein the catalytically active material comprises a plurality of aluminium oxide beads.

11. (canceled)

12. A reactor according to claim 1, wherein the one or more hydrocarbon(s) are selected from the group consisting of formic acid, acetic acid and oxalic acid.

13. A reactor system comprising:
two or more plasma-bubble reactors according to claim 1 and
a plurality of fluid conduits, wherein each fluid conduit is configured to operably couple adjacent plasma-bubble reactors together via corresponding ports to enable fluid communication of one or more of CO₂ gas, H₂O₂, one or more hydrocarbon(s), syngas and/or H₂O therebetween.

14. (canceled)

15. (canceled)

16. A reactor system according to claim 13, further comprising one or more of the following:
(i) a pump for fluidly communicating water from a water supply to the vessel of one of the two or more plasma-bubble reactors;
(ii) a compressor for enhancing the flow of CO₂ gas from the input feed to the vessel of one of the two or more plasma-bubble reactors;
(iii) a flowmeter disposed in line between the compressor and the vessel of the one plasma-bubble reactor to monitor the flow rate of the CO gas; and/or
(iv) a liquid receiver for receiving H₂O; from the vessel of one of the two or more plasma-bubble reactors.

17. (canceled)

18. (canceled)

19. (canceled)

20. (canceled)

21. A method for producing hydrogen peroxide (H₂O₂), one or more hydrocarbon(s), and syngas, the method comprising the steps of:
generating plasma from an input feed comprising carbon dioxide (CO₂) gas to produce an activated CO₂ gas encapsulated within a plurality of bubbles formed in liquid; and
reacting the activated CO₂ gas with water (H₂O) at a plasma-liquid interface formed between the bubbles and the surrounding liquid to produce hydrogen peroxide (H₂O₂), one or more hydrocarbon(s) and syngas.

22. A method according to claim 21, wherein the plasma is generated by applying a potential difference across two electrodes, wherein at least one of the two electrodes is a high voltage (HV) electrode at least partially immersed within the liquid, and configured to generate an electric discharge through the liquid for activating the CO₂ gas encapsulated within the bubbles.

23. (canceled)

24. A method according to claim 22, wherein the potential difference falls within a range of between about 1 kV and about 100 kV.

25. A method according to claim 21, wherein the liquid is an aqueous medium, and wherein the aqueous medium comprises an electrolyte.

26. (canceled)

27. A method according to claim 21, wherein the reaction is carried out in a vessel substantially under atmospheric pressure and room temperature.

28. A method according to claim 21, wherein the input feed comprises a mixture of the CO₂ gas and a second gas selected from the group consisting of carbon monoxide (CO), water vapour/steam (H₂O), methane (CH₄), hydrogen (H₂), nitrogen (N₂) and any mixture thereof.

29. (canceled)

30. A method according to claim 22, wherein the HV electrode is partially enclosed within a tube defining a gas passage extending partially along a length of the HV electrode, the method further comprising the step of:
adjusting the vertical position of the HV electrode relative to the vertical position of the tube to generate longer plasma streamers within the gas passage.

31. (canceled)

32. A method according to claim 21, further comprising the step of:
catalysing the reaction between the activated CO₂ gas and H₂O.

33. A method according to claim 21, wherein the one or more hydrocarbon(s) are selected from the group consisting of formic acid, acetic acid and oxalic acid.
